(19) United States
(12) Patent Application Publication
(10) Pub. No.: US 2010/0228195 A1
(43) Pub. Date: Sep. 9, 2010

(54) Needle puncture device for medical equipment

(75) Inventors: Ulrich KEHR, Gaetringen (DE); Frank SCHWENKER, Kirchheim unter Teck (DE); Andreas WEBER, Stuttgart (DE); Andreas NESCH, Nagold (DE)

Correspondence Address: COLLARD & ROE, P.C., 1077 NORTHERN BOULEVARD, ROSLYN, NY, 11576, US

(73) Assignee: Dr. Fritz Faulhaber GmbH & Co. KG

(21) Appl. No.: 12/584,125

(22) Filed: Aug. 31, 2009

Related U.S. Application Data
(60) Provisional application No. 61/210,367, filed on Mar. 18, 2009.

(30) Foreign Application Priority Data
Mar. 6, 2009 (DE) 20 2009 003 050.4

(51) Int. Cl. A61M 37/00

(52) U.S. Cl. 604/131

(57) ABSTRACT

The present invention relates to a needle puncture device (1) for medical equipment, in particular for an injection unit, having a drive element (2) for a needle (4) which can be driven by drive means (6) for a puncture movement. The drive means (6) are designed to transfer movement by means of a cam mechanism (8).

### CROSS REFERENCE TO RELATED APPLICATIONS

Applicants claim priority under 35 U.S.C. §119(e) of U.S. Provisional Application No. 61/210,367 filed Mar. 18, 2009. Applicants also claim priority under 35 U.S.C. §119 of German Application No. 20 2009 003 050.4 filed Mar. 6, 2009.

The present invention relates to a needle puncture device for driving a needle, in particular an injection needle (cannula), or a needle support within a piece of medical equipment, the device having a drive element which can be driven by drive means for a needle-puncture movement and preferably also for a subsequent needle-retraction movement.

Injection devices are known, by means of which a user (patient) can self-administer certain medication such as insulin. In general, the following factors have to be taken account of in the process:
the safety of the user
ensuring a minimal puncturing force of the needle
the puncturing process and the retraction lift should be carried out within a timeframe which is as short as possible
low noise
compact installation space
low energy use

WO 2005/097237 A1 describes such a piece of injection equipment. This battery-operated piece of equipment comprises a cartridge with a number of cannulae (injection needles), a container with the medication to be administered and a puncture device of the above-described, generic type. In order to administer the medication, respectively one cannula must be guided, in a short period of time and with a certain amount of force, from the cartridge firstly to the medication container and secondly under the skin. In the process, both a sealing foil of the needle stored in the cartridge and a membrane in the medication container have to be pierced, with a certain minimum force being required for this purpose. The known puncture device, which is described to this end in the document with reference to FIGS. 54 to 58, is a fairly complex design of a large number of individual components, with the actual lifting movements of the drive element for injecting and retracting the needle being effected by pressure springs. These pressure springs are respectively tensed by an electric drive motor and a relatively complicated mechanism (transmission with cam disk) and released for the respective movement by releasing a lock. Although the spring drive can achieve the—in principle desirable—fast movements, in particular in the needle puncture device, the known device does however have a few disadvantages in addition to the complicated design. The main disadvantage is that the excess spring energy has to be absorbed in each case by a mechanical stop, which leads to irritating noises. Furthermore, residual oscillations of the spring-mass system can lead to undesired needle movement which is very unpleasant for the user or can even cause pain. After the puncture lift effected by the spring force, the retraction lift has to first of all be prepared by tensing the resetting spring by motor-driven rotation of the cam disk. This leads to a waiting time for the user after an injection which is found to be particularly irritating because the needle is still located in the body of the user during this period. The mechanical stops at the end positions lead to very irritating noises because large masses have to be moved in the known mechanism. Furthermore, sufficiently stable structures are required in this case to absorb the spring forces in the device even over a relatively long period of time. Moreover, there is no guarantee that the needle is always retracted after an injection, if, for example, the voltage supply of the piece of equipment should fail before the retraction.

The document US 2007/0066938 A1 describes a further piece of injection equipment, in which a direct drive is respectively provided for both the needle drive and the medication injection by means of leadscrews. The puncturing and retraction take a long time as a result of the spindle drives. Spindle drives are also disadvantageous with respect to the safety aspect because, in the case of a fault, in particular a voltage failure, the spindle drives are blocked due to self-locking.

The publications EP 1 669 028 B1, EP 1 792 568 A1 and EP 1 970 007 A1 each describe a lancet device by means of which a small puncture wound for taking a blood sample (e.g. for blood-sugar measurements) is intended to be produced by a short, fast needle puncturing movement.

The present invention is based on the object of providing a needle puncture device of the described, generic type which ensures optimized kinematics, increased safety during use and improved usage comfort by means of simple design means.

According to the invention, this is achieved by the features of the independent Claim 1. Advantageous features of refinements are contained in the dependent claims and in the subsequent description.

Accordingly, according to the invention, the drive means—in particular in the form of a motor-driven direct drive—are designed with direct movement transfer by means of a cam mechanism. Since a cam mechanism is, by definition, a transmission with an uneven transmission ratio, a specific design of a radial cam makes it possible to almost arbitrarily design the movement of a scanning element interacting with the radial cam, and hence also to design the movement of the drive element connected to the scanning element by, in particular, a toothed transmission. As a result, the kinematics of the needle puncture device according to the invention can be optimized by a simple design and in an economic fashion. In an advantageous refinement, provision can also be made for such a design in which the drive means act as a kinetic energy store. To this end, provision is made in a preferred refinement for the radial cam, which is formed on a control rotor to be motor-driven in a rotating fashion, to have at least one acceleration region in which a rotation of the control rotor does not effect movement of the scanning element or the drive element. Hence, this acceleration region can be used for accelerating or decelerating (negative acceleration) the control rotor and all other rotating components, that is to say the whole rotating mass system (inertial-/flywheel mass), without the drive element and the injection needle moving. In this case, kinetic energy is conveniently stored as a result of the rotation of the mass system in the acceleration region. This kinetic energy of the accelerated mass system acting as a flywheel mass is partly emitted when the actual work movement is carried out. This makes it possible to dimension the drive to be smaller and more compact. It is possible to decelerate the control rotor or the mass system in a defined manner, without moving the drive element and before it reaches a final position, by means of a further acceleration or deceleration region of the radial cam. The medication is dosed or injected once this position has been reached. The radial cam is run through in the reverse direction for the subsequent return lift, the deceleration region then forming an acceleration region and the acceleration region then forming a deceleration region.

The invention will be explained in more detail with reference to an advantageous refinement and a preferred exemplary embodiment illustrated in the drawing, in which

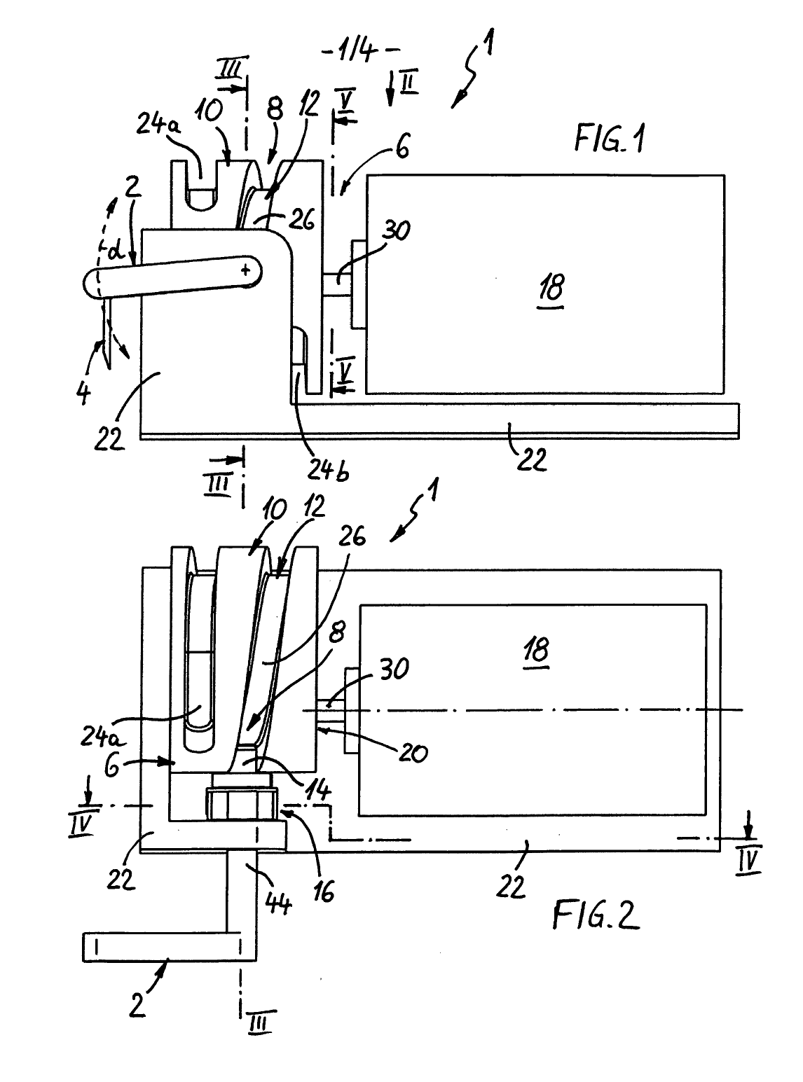

FIG. 1 shows a side view of a needle puncture device according to the invention,

FIG. 2 shows a plan view of the device in the direction of the arrow II in accordance with FIG. 1,

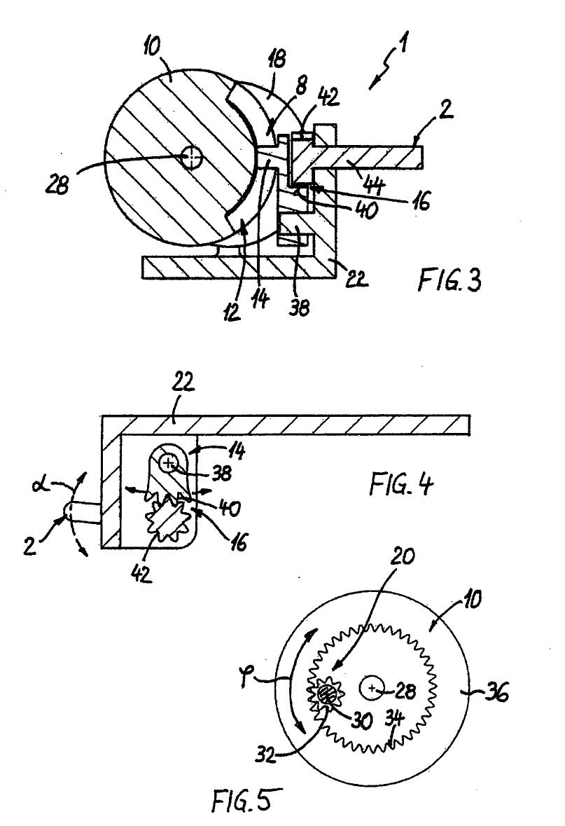

FIG. 3 shows a cross section in the plane III-III in accordance with FIG. 1 and FIG. 2,

FIG. 4 shows a section according to the cut line IV-IV in FIG. 2,

FIG. 5 shows a sectional view of the control rotor in the plane V-V in accordance with FIG. 1,

FIG. 6 shows a reduced perspective exploded view of the needle puncture device according to the invention in accordance with FIGS. 1 to 5,

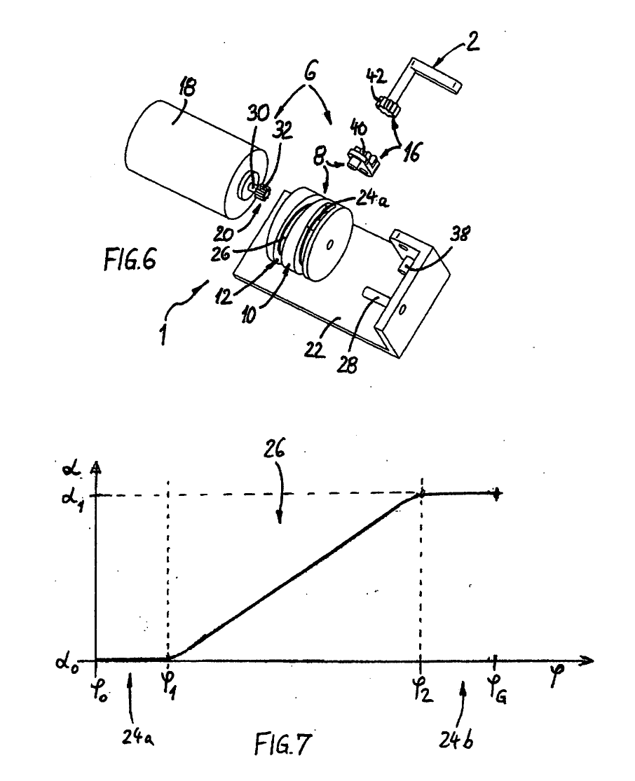

FIG. 7 shows a diagram to explain the drive kinematics of the puncture device according to the invention,

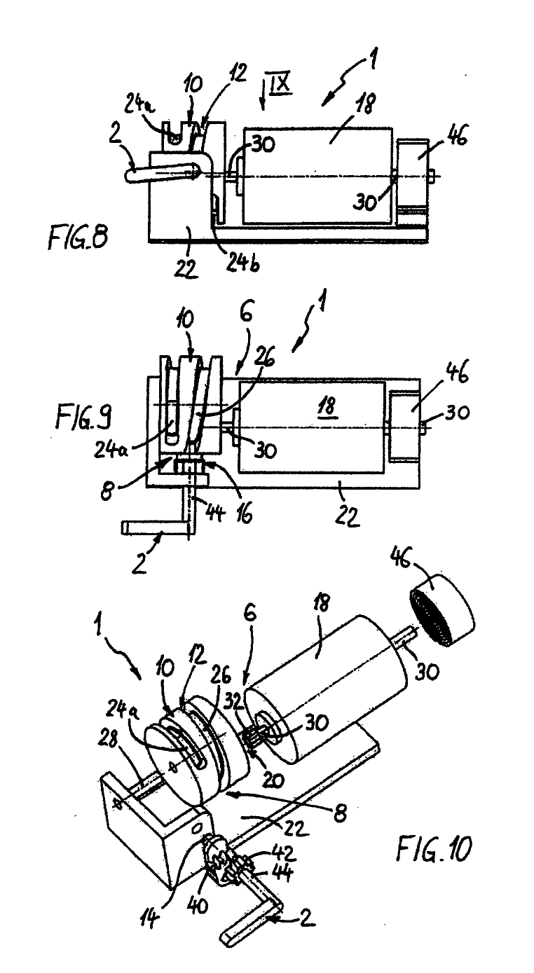

FIG. 8 shows a reduced view similar to FIG. 1 in an advantageous refinement of the puncture device according to the invention,

FIG. 9 shows a view analogous to FIG. 2 in the direction of the arrow IX in accordance with FIG. 8, and

FIG. 10 shows a perspective exploded view of the embodiment in accordance with FIGS. 8 and 9.

In the various figures of the drawing, the same parts are always provided with the same reference symbol.

A needle puncture device 1 according to the invention is preferably used to drive an injection needle within a piece of medical injection equipment. To this end, the puncture device 1 has a drive element 2. FIG. 1 indicates an injection needle 4 which is directly connected to the drive element 2. However, in practice, this will usually be an indirect connection, with the injection needle 4 intended to be connected to the drive element 2 by means of parts which are not illustrated. The drive element 2 can be driven by drive means 6 for a needle puncture movement and preferably also for an opposite needle retraction movement.

According to the invention, the drive means 6 are designed as a motor-driven, in particular rotational, direct drive with a direct movement transfer by means of a cam mechanism 8. The cam mechanism 8 comprises a control rotor 10, which is intended to be motor-driven in a rotating fashion, with a radial cam 12 and a scanning element 14 which can be moved in a cam-like fashion over the profile of the radial cam 12. In the process, the movement of the scanning element 14 is transferred to the drive element 2, in particular via a toothed transmission 16. However, it is also possible to connect the scanning element 14 directly to the drive element 2, or to design the drive element 2 itself as a scanning element. Furthermore, the drive means 6 have an electric motor 18 which drives the control rotor 10, preferably via a toothed transmission 20. All components of the puncture device 1 are held or mounted on a base part 22.

In a preferred embodiment of the invention, the drive means 6 are designed as a kinetic energy store. To this end, the radial cam 12 has at least one acceleration region 24 in which a rotation of the control rotor 10 does not effect movement of the scanning element 14, and hence it does not effect movement of the drive element 2 either. In the illustrated, preferred embodiment, the radial cam has one movement region 26 with, in particular, an approximately linear gradient profile of the needle puncture and retraction movements. Respectively one acceleration region 24a, 24b adjoins this movement region 26 of the radial cam 12, preferably on both sides.

In this respect, reference is made to the diagram shown in FIG. 7. Therein, the rotation of the control rotor 10 through the rotational angle φ is illustrated and, as a function of this rotational angle φ, the movement of the drive element 2 is illustrated, in an exemplary manner, as a pivot movement through an angle α. Before a puncturing process, the drive element 2 is first of all in a position α₀. In the case of a rotation of the control rotor 10, the drive element 2 first of all remains in this initial position α₀ as a result of the first acceleration region 24a of the radial cam 12. It follows that the control rotor 10 can first of all be accelerated through this rotational angle from φ₀ to φ₁ without the drive element 2 moving such that the rotating mass system stores kinetic energy. In the case of further rotation of the control rotor 10, from the angle φ₁ to φ₂, the drive element 2 is moved from its initial position α₀ to its puncturing position α₁ by means of the scanning element 14. Advantageously, the previously stored kinetic energy is again emitted at least in part during this movement. This makes it possible to perform the puncturing process very quickly, but advantageously without jolts. In the case of further rotation of the control rotor 10, from the position φ₂ to the final position φ_{G}, the rotating mass system is decelerated over the second acceleration region 24b without further movement of the drive element 2. Reverse movement to retract the needle is correspondingly effected in the reverse order.

The drive means 6 according to the invention can, for example, be designed such that a complete rotation of the control rotor 10 through a rotational angle from φ₀ to φ_{G} is effected over 540°. The actual movement region 26 extends from the angle from φ₁ to φ₂, in particular over 300°. The/each acceleration region 24 or 24a, 24b extends over an angle of preferably 120°. In this case, the drive means 6 can be designed for a pivoting movement of the drive element 2, to and fro, through an angle α in the range from 60° to 100°, in particular approximately 90°.

In a further preferred refinement, the control rotor 10 is of cylindrical design and is rotatably mounted on a bearing journal 28 (see FIGS. 6 and 10) of the base part 22. The radial cam 12 is preferably designed as a groove that is open radially outward on the outer cylinder circumference of the control rotor 10. The movement region 26 of the radial cam 12 runs along the circumference of the control rotor 10 like a helix with a certain gradient. The movement region 26 merges into the acceleration regions 24a, b in a continuous manner, with each acceleration region 26a, b running in the circumferential direction with a gradient of zero above the control rotor 10. The electric motor 18 has on its drive shaft 30 a drive cog 32 which meshes with toothing 34 of the control rotor 10. As can be seen in particular in FIG. 5, the toothing 34 is preferably arranged on the inner circumference of a hollow cylinder wall 36 of the control rotor 10. The motor 18 thus axially meshes with the control rotor 10 by means of the drive cog 32.

In the illustrated embodiments, the scanning element 14 is designed as a lever mounted such that it can pivot on a bearing journal 38 of the base part 22. However, in principle it is also possible to design the scanning element 14 as a slider guided such that it can move linearly. In accordance with FIG. 4, in both cases it is possible for the scanning element 14 to have a toothing 40 which meshes with a complementary toothing 42 on a pivot shaft 44 of the drive element 2. In this case, the drive element 2 is also rotatably mounted in a corresponding mounting opening of the base part 22 by means of its pivot shaft 44.

As a result of the refinement described above, the scanning element 14 follows the profile of the radial cam 12 when the control rotor 10 rotates, with it moving to and fro over the movement region 26 in accordance with the rotational axis, or parallel to the latter. This results in a rotation or pivoting of the pivot shaft 44 about an axis basically perpendicular to the axis of rotation of the control rotor 10.

The electric motor 18 can preferably be designed such that it acts as a generator when the drive means 6 are decelerating such that energy obtained during the deceleration can be fed back into the electricity supply system of the respective piece of equipment (e.g. charging a capacitor, operating a different equipment function). To this end, the motor 18 may be in the form of a bell-type armature motor, a coreless flat rotor or the like.

As illustrated by way of example in FIGS. 8 to 10, the drive means 6 can have an additional resetting clockwork 46 which is either always prestressed or is tensed during a puncture movement in order to release its energy for a retraction movement where necessary. This advantageous refinement increases the safety in the case of a power cut in the battery voltage supply, with the resetting clockwork 46 in any case moving the drive element 2 back into the position in which the injection needle is retracted. To this end, the clockwork 46 is preferably connected to an end of the drive shaft 30 of the electric motor 18 which is remote from the control rotor 10 and is designed like a winding spring for a clock (spiral spring).

Furthermore, in the illustrated examples, the radial cam 12 of the control rotor 10 is designed for a reversal of rotational direction between puncture and retraction movements. Alternatively, a refinement with a revolving, self-contained profile of the radial cam 12 for rotating the control rotor 10 in only one direction, that is to say without a reversal of rotational direction, is also possible. A design where the radial cam 12 is arranged on an end face of the control rotor 10 is also feasible. Furthermore, as an alternative to the illustrated embodiment, the control rotor 10 can also be designed like a disk.

The invention is not limited to the illustrated and described exemplary embodiments; rather it comprises all embodiments with an equivalent effect in the sense of the invention. Thus, it is also possible to use a (rotary) clockwork as an alternative to the electric motor. Additionally, the control rotor and the motor can be combined to form a drive component (without transmission). Furthermore, provision can in principle be made for a cam mechanism with a non-rotational but, for example, linearly-moved drive. The invention is suitable not only for injection equipment, but also for equipment for taking blood samples and for equipment for generating small puncture wounds (so called lancet devices).

Moreover, the invention is in any case not restricted to the feature combination defined in Claim 1, but rather it can also be defined by any other arbitrary combination of certain features of all individual features disclosed overall. This means that, in principle, practically every individual feature of Claim 1 can be omitted or can be replaced by at least one individual feature disclosed at a different point in the application. In this respect, Claim 1 is to be understood to only be a first attempt at phrasing an invention.

1. Needle puncture device (1) for medical equipment, in particular for an injection unit, having a drive element (2) for a needle (4) which can be driven by drive means (6) for a puncture movement, wherein the drive means (6) are designed to transfer movement by means of a cam mechanism (8).

2. Needle puncture device according to claim 1, wherein the drive means (6) are designed as a kinetic energy store.

3. Needle puncture device according to claim 1, wherein the drive means (6) can also drive the drive element (2) in order to perform a needle retraction movement.

4. Needle puncture device according to claim 1, wherein the cam mechanism (8) comprises a control rotor (10), which is intended to be motor-driven in a rotating fashion, with a radial cam (12) and a scanning element (14), which can be moved in a cam-like fashion over the profile of the radial cam (12), the movement of the scanning element (14) being transferred onto the drive element (2).

5. Needle puncture device according to claim 4, wherein the radial cam (12) has at least one acceleration region (24) in which a rotation of the control rotor (10) does not effect movement of the scanning element (14) or the drive element (2).

6. Needle puncture device according to claim 4, wherein the radial cam (12) has at least one movement region (26) with, in particular, an approximately linear gradient profile of the needle puncture/retraction movement.

7. Needle puncture device according to claim 5, wherein respectively one acceleration region (24a; 24b) adjoins the movement region (26) of the radial cam (12) on both sides.

8. Needle puncture device according to claim 4, wherein the control rotor (10) is of cylindrical design, the radial cam (12) being designed as a groove on the outer cylinder circumference.

9. Needle puncture device according to claim 4, wherein the drive means (6) have an electric motor (18) which preferably drives the control rotor (10) by means of a toothed transmission (20).

10. Needle puncture device according to claim 9, wherein the electric motor (18) has on its drive shaft (30) a drive cog (32) which meshes with toothing (34) of the control rotor (10), the toothing (34) preferably being arranged on the inner circumference of a hollow cylinder wall (36) of the control rotor (10).

11. Needle puncture device according to claim 1, wherein the drive element (2) is designed as a pivoted lever, the drive means (6) being designed such that the drive element (2) for the puncture/retraction movement can be moved back and forth over an angle α in a range of 60° to 100°, in particular of approximately 90°.

12. Needle puncture device according to claim 4, wherein the scanning element (14) is designed as a pivotably mounted lever or a linearly movable slider and connected to the drive element (2), in particular by means of a toothed transmission (16).

13. Needle puncture device according to claim 1, wherein the drive means (6) have a prestressed resetting clockwork (46), which releases its energy for a retraction movement where necessary.

14. Needle puncture device according to claim 13, wherein the resetting clockwork (46) is connected to the drive shaft (30) of the electric motor (18) and is designed like a winding spring for a clock.

15. Needle puncture device according to claim 3, wherein the radial cam (12) of the control rotor (10) is designed either with two end regions for a reversal of rotational direction between puncture and retraction movements or with a revolving, self-contained profile for rotation without a reversal of direction.
